(19) United States
(12) Patent Application Publication
(10) Pub. No.: US 2010/0331264 A1
(43) Pub. Date: Dec. 30, 2010

(54) PRESERVING SECONDARY PEPTIDE STRUCTURE

(76) Inventors: Thitiwan Buranachokpaisan, Bedminster, NJ (US); Feng Liu, Succasunna, NJ (US)

Correspondence Address: NOVARTIS;CORPORATE INTELLECTUAL PROPERTY, ONE HEALTH PLAZA 101/2, EAST HANOVER, NJ, 07936-1080, US

(21) Appl. No.: 12/668,167

(22) PCT Filed: Jul. 9, 2008
(86) PCT No.: PCT/US08/69456

§ 371 Date: Jan. 7, 2010

Related U.S. Application Data
(60) Provisional application No. 60/948,525, filed on Jul. 9, 2007.
(60) Provisional application No. 60/955,480, filed on Aug. 13, 2007.

(51) Int. Cl. A61K 38/10

(52) U.S. Cl. 514/21.4

(57) ABSTRACT

A method of preserving the α-helix secondary structure of N-Acetyl-D-Asp-D-Trp-D-Phe-D-Lys-D-Ala-D-Phe-D-Tyr-D-Asp-D-Lys-D-Val-D-Ala-D-Glu-D-Lys-D-Phe-D-Lys-D-Glu-D-Ala-D-Phe-Amide or N-Acetyl-L-Asp-L-Trp-L-Phe-L-Lys-L-Ala-L-Phe-L-Tyr-L-Asp-L-Lys-L-Val-L-Ala-L-Glu-L-Lys-L-Phe-L-Lys-L-Glu-L-Ala-L-Phe-Amide and compositions comprising such peptides are disclosed.

### BACKGROUND OF THE INVENTION

1. Field of the Invention

The present invention is directed to a method of preserving the α-helix secondary structure of certain peptides during freeze-drying, as well as to freeze-dried formulations of such peptides made according to the method.

2. Related Background Art

APP018 and APL180 are known apolipoprotein (apo) A-I mimetics and are disclosed in U.S. Pat. Nos. 6,664,230 and 6,933,279 and WO 2004/034977, respectively. Each of these peptides comprises an 18 amino acid sequence, namely D-W-F-K-A-F-Y-D-K-V-A-E-K-F-K-E-A-F (Ac-Asp-Trp-Phe-Lys-Ala-Phe-Tyr-Asp-Lys-Val-Ala-Glu-Lys-Phe-Lys-Glu-Ala-Phe-NH₂—SEQ ID NO 1), having an acetyl amino-terminal protecting group and an amide carboxyl-terminal protecting group. In free (unbound) form, when all amino acids are in D-form, the peptide is known as APP018; when all the amino acids are in L-form, the free (unbound) peptide is known as APL180. These peptides have four phenylalanines and are sometimes referred to as “D4F” when the amino acids are all in the D form or “L4F” when the amino acids are all in the L form. Reverse “4F” is a mirror image of 4F with the relative positions of the amino acids to each other and to the hydrophilic and hydrophobic faces being identical. Similarly, peptides in this group contain two phenylalanines, known as 2F, three phenylalanines, known as 3F, five phenylalanines, 5F, six phenylalanines, 6F and seven phenylalanines or 7F. It is possible to have mirror images or reverse peptides based on these peptides also.

All these peptides, have been shown to inhibit low density lipoprotein (LDL) oxidation, stimulate reverse cholesterol transport, and reduce formation of atherosclerotic lesion. Accordingly, these agents are useful in the treatment of cardiovascular disease which remains a leading cause of morbidity and mortality, particularly in the United States and in Western European countries. Hence, effective formulation of these peptides is highly desirable.

Exchangeable apolipoproteins, including apo A-I, possess lipid-associating domains (Brouillette at al., Biochim. Biophys. Acta 1256:103-129 (1995); Segrest et al., FEBS Lett. 38::247-253 (1974)). Apo A-I has been postulated to possess eight tandem repeating 22 mer sequences. Characteristics of the class A amphipathic helix include the presence of positively charged residues at the polar-nonpolar interface and negatively charged residues at the center of the polar face (Id.; Segrest et al., Proteins: Structure, Function, and Genetics 8: 103-117 (1990)). Apo A-I has been shown to strongly associate with phospholipids to form complexes and to promote cholesterol efflux from cholesterol-enriched cells. It has now been shown that the secondary structure of apo A-I is essential for high affinity binding to lipids, ultimately leading to its biological activity (Saito et al., J. Biol. Chem. 279(20): 20974-20981 (2004)). Hence, preservation of the secondary structure of apo A-I is highly desirable. Without limiting the invention to a particular mechanism of action, it may be that preservation of the α-helix conformation may be necessary for is important for giving apo-I its binding affinity to lipids.

The invention of U.S. Pat. No. 6,664,230 which provided novel peptides comprising 18 amino acids having a class A amphipathic helix when formulated with “D” amino acid residue(s) and/or having protected amino and carboxyl termini which when orally administered to an organism, are readily taken up and delivered to the serum, and are effective to mitigate one or more symptoms of atherosclerosis.

Freeze-drying proteins is a common approach to improve both chemical and physical stability of the protein. However, freezing and dehydration stress can cause protein aggregation, leading to a loss of it's bioactivity. Trehalose, α-D-glucopyranosyl-α-D-glucopyranoside, is a naturally occurring disaccharide, which has been shown to be useful in preventing denaturation of proteins and other macromolecules, viruses and foodstuffs during drying processes. See, e.g., U.S. Pat. Nos. 4,891,319, 5,149,653, 5,026,566, 5,902,565 and 6,890,512. EP 0 762 897, while indicating that the method of preventing aggregation disclosed therein is applicable to both proteins and peptides, exemplifies its method with human growth hormone only. Trehalose has also been extensively studied as a protein stabilizer in the literature (Kaushik et al., J. Bio. Chem. 278 (29): 26458-26465 (2003)). To date, no suggestion that trehalose may be effective in preserving peptide secondary structure has been noted in the prior art.

Accordingly, a method of preserving the α-helix (secondary) structure of APP018 and APL180 during freeze-drying by trehalose would be desirable.

### SUMMARY OF THE INVENTION

The present invention is directed to a method of preserving secondary structure during freeze-drying of a peptide comprising the steps of: (a) admixing trehalose with the peptide in a solution, said trehalose in an amount sufficient to preserve secondary structure of the peptide; and (b) freeze-drying the solution or suspension to obtain a peptide composition in which secondary structure has been preserved, wherein the peptide is selected from N-Acetyl-D-Asp-D-Trp-D-Phe-D-Lys-D-Ala-D-Phe-D-Tyr-D-Asp-D-Lys-D-Val-D-Ala-D-Glu-D-Lys-D-Phe-D-Lys-D-Glu-D-Ala-D-Phe-Amide (D4F); N-Acetyl-L-Asp-L-Trp-L-Phe-L-Lys-L-Ala-L-Phe-L-Tyr-L-Asp-L-Lys-L-Val-L-Ala-L-Glu-L-Lys-L-Phe-L-Lys-L-Glu-L-Ala-L-Phe-Amide (L4F), D3F, L3F, D5F, L5F, D6F, L6F, D7F and L7F or any pharmaceutically acceptable salt form thereof. In a preferred embodiment of this aspect of the invention, the peptide is L4F. The invention is further directed to a method further comprising the step of: (c) reconstituting the peptide composition to obtain a solution or suspension of the peptide in which secondary structure has been preserved.

In certain preferred embodiments of the invention, the secondary structure is an α-helix structure. In other preferred embodiments, the solution or suspension of step (a) further comprises at least one additional freeze-drying excipient such as buffer or surfactant.

The present invention is further directed to freeze-dried and reconstituted compositions made according to the method of the invention.

The present invention is still further directed to a freeze-dried composition comprising a peptide and an amount of trehalose sufficient to preserve secondary structure of the peptide, wherein the peptide is selected from N-Acetyl-D-Asp-D-Trp-D-Phe-D-Lys-D-Ala-D-Phe-D-Tyr-D-Asp-D-Lys-D-Val-D-Ala-D-Glu-D-Lys-D-Phe-D-Lys-D-Glu-D-Ala-D-Phe-Amide; N-Acetyl-L-Asp-L-Trp-L-Phe-L-Lys-L-Ala-L-Phe-L-Tyr-L-Asp-L-Lys-L-Val-L-Ala-L-Glu-L-Lys-L-Phe-L-Lys-L-Glu-L-Ala-L-Phe-Amide, D3F, L3F, D5F, L5F, D6F, L6F, D7F and L7F or any pharmaceutically acceptable salt form thereof.

### DETAILED DESCRIPTION

The present invention is directed to a method of preserving secondary structure during freeze-drying of a peptide. As used herein, “secondary structure” refers to the general three-dimensional form of biomolecules such as peptides or of segments of biomolecules, such as proteins and nucleic acids; for purposes of the present invention, “secondary structure” preferably refers to the α-helix structure of certain peptides. As used herein, “preserving” (and other forms thereof) refers to keeping intact. Preserving preferably refers to maintainence or improvement (increase) of the α-helix content of certain peptides—in other words, the α-helix content of a particular freeze-dried composition made according to the method of the present invention will be greater than that of a freeze-dried composition made according to conventional processes. As used herein, “freeze-drying” (and other forms thereof) refers to any process by which water is removed from a material which is first frozen and then subjected to reduced pressure and/or heat which allows the water to sublime directly from the solid phase to gas.

More specifically, the first embodiment of the present invention comprises the steps of: (a) admixing trehalose with the peptide in a solution or suspension, said trehalose in an amount sufficient to preserve secondary structure of the peptide; and (b) freeze-drying the solution or suspension to obtain a peptide composition in which secondary structure has been preserved, wherein the peptide is selected from N-Acetyl-D-Asp-D-Trp-D-Phe-D-Lys-D-Ala-D-Phe-D-Tyr-D-Asp-D-Lys-D-Val-D-Ala-D-Glu-D-Lys-D-Phe-D-Lys-D-Glu-D-Ala-D-Phe-Amide; N-Acetyl-L-Asp-L-Trp-L-Phe-L-Lys-L-Ala-L-Phe-L-Tyr-L-Asp-L-Lys-L-Val-L-Ala-L-Glu-L-Lys-L-Phe-L-Lys-L-Glu-L-Ala-L-Phe-Amide, D3F, L3F, D5F, L5F, D6F, L6F, D7F and L7F or any pharmaceutically acceptable salt form thereof. Preferably, the peptide is the free form. N-Acetyl-L-Asp-L-Trp-L-Phe-L-Lys-L-Ala-L-Phe-L-Tyr-L-Asp-L-Lys-L-Val-L-Ala-L-Glu-L-Lys-L-Phe-L-Lys-L-Glu-L-Ala-L-Phe-Amide. The individual peptides are generally referred to herein as a peptides of the invention.

In the first step of the inventive method, trehalose is admixed with a peptide of the invention in a solution.

Trehalose is a commercially available material and can be purchased from any source. Either the N-Acetyl-D-Asp-D-Trp-D-Phe-D-Lys-D-Ala-D-Phe-D-Tyr-D-Asp-D-Lys-D-Val-D-Ala-D-Glu-D-Lys-D-Phe-D-Lys-D-Glu-D-Ala-D-Phe-Amide; N-Acetyl-L-Asp-L-Trp-L-Phe-L-Lys-L-Ala-L-Phe-L-Tyr-L-Asp-L-Lys-L-Val-L-Ala-L-Glu-L-Lys-L-Phe-L-Lys-L-Glu-L-Ala-L-Phe-Amide peptide, or any peptide of the invention, can be purchased from commercial sources or made according to known procedures as described in U.S. Pat. Nos. 6,664,230 and 6,933,279 and PCT International Publication No. WO 2004/034977, the entire disclosure of each of which is incorporated by reference herein.

Trehalose and a peptide of the invention are admixed in water. According to the present invention, trehalose may be added to a solution of a peptide of the invention, the peptide may be added to a solution of trehalose or both trehalose and the peptide may be added to a solvent to form a solution in step (a). The pH is adjusted to a range of from about 3 to 11, more preferably 6 to 9, even more preferably 6.5 to 9. Preferably a surfactant, including but not limited to TWEEN 80, is added prior to the addition of the peptide, TWEEN 80 is present in an amount ranging preferably from about 0.0001% to about 10% weight by volume.

The amount of trehalose sufficient to preserve secondary structure of the peptide corresponds to a range preferably from about 1 to about 50%, more preferably from about 10 to 25% weight by volume, with about 10% weight by volume being most preferred. This corresponds to a weight ratio of trehalose to peptide range of from about 500:0.01 to about 10:200, preferably of from about 250:0.2 to about 100:30 and most preferably about 100:0.2 to about 100:30.

Admixing can be accomplished by any conventional means, i.e., simple mixture.

In a preferred embodiment of the present invention, the solution of step (a) further comprises at least one additional buffer. Buffers suitable for use in the present invention include, without limitation, sodium phosphate, for example mono or di sodium phosphate, potassium phosphate, Tris, citrate, tartrate and histidine and combinations thereof. When present, phosphate buffer concentration corresponds to a range preferably from about 1 mM to about 1 M of the solution of step (a), preferably from about 5 mM to about 100 mM.

In the second step of the inventive method, the solution or suspension is freeze-dried to obtain a peptide composition in which secondary structure has been preserved. Freeze-drying can be accomplished by any known means. For example, freeze-drying may involve the use of a freeze-drying flask which is rotated in a bath, which is cooled by mechanical refrigeration, dry ice and methanol, or liquid nitrogen or may involve the use of a large-scale freeze-drying machine. As a result of freeze-drying the combination of trehalose and peptide, the secondary structure of the peptide in the peptide composition will have been preserved. In other words, the peptide composition of step (b) has a high α-helix content as compared to a peptide composition which was freeze-dried without the use of trehalose.

An optional step for the first embodiment of the invention comprises (c) reconstituting the peptide composition to obtain a solution of the peptide in which secondary structure has been preserved. Reconstitution can be accomplished by any known means such as by the simple addition of water to the peptide composition of step (b). As one of ordinary skill in the art will readily appreciate, solutions of varying peptide concentration can be achieved by reconstitution with varying amounts of solvent. Solvents suitable for use in step (c) include, without limitation, water, buffer solution or isotonic solution. As a result of the reconstitution of the freeze-dried peptide composition, the secondary structure of the peptide will have been preserved. In other words, the solution of step (c) has a high peptide secondary structure content, and possibly a high α-helix content as compared to a solution or suspension which was reconstituted from a freeze-dried composition which did not use trehalose in accordance with this invention.

Additional embodiments of the invention are directed to freeze-dried composition and reconstituted compositions made according to the method of the first embodiment of the invention.

Yet another embodiment of the invention is directed to a freeze-dried composition comprising a peptide which is N-Acetyl-D-Asp-D-Trp-D-Phe-D-Lys-D-Ala-D-Phe-D-Tyr-D-Asp-D-Lys-D-Val-D-Ala-D-Glu-D-Lys-D-Phe-D-Lys-D-Glu-D-Ala-D-Phc-Amide; N-Acetyl-L-Asp-L-Trp-L-Phe-L-Lys-L-Ala-L-Phe-L-Tyr-L-Asp-L-Lys-L-Val-L-Ala-L-Glu-L-Lys-L-Phe-L-Lys-L-Glu-L-Ala-L-Phe-Amide, D3F, L3F, D5F, L5F, D6F, L6F, D7F and L7F or any pharmaceutically acceptable salt form thereof and an amount of trehalose sufficient to preserve the secondary structure of the peptide, Details regarding the amounts of peptide and trehalose are the same as those noted above with regard to the first embodiment of the invention.

Specific embodiments of the invention will now be demonstrated by reference to the following examples. It should be understood that these examples are disclosed solely by way of illustrating the invention and should not be taken in any way to limit the scope of the present invention.

### Example 1

Freeze-dried compositions of N-Acetyl-L-Asp-L-Trp-L-Phe-L-Lys-L-Ala-L-Phe-L-Tyr-L-Asp-L-Lys-L-Val-L-Ala-L-Glu-L-Lys-L-Phe-L-Lys-L-Glu-L-Ala-L-Phe-Amide (APL180) were made using the ingredients noted in Table 1 below.

TABLE 1

| | | | | | | | | Filled | | |
|---|---|---|---|---|---|---|---|---|---|---|
| | | | | Disodium | Disodium | | | volume | | APL180 |
| Formulation | | Trehalose | Trehalose | phosphate | Phosphate | TWEEN | TWEEN | per vial | Reconstituted | (mg/ml) after |
| # | APL180 | (mg) | (%) | (mg) | (%) | 80 (mg) | 80 (%) | (ml) | volume (ml) | reconstitution |
| 1 | 1 mg | 100 | 10 | 2.1 | 0.21 | | | 1 | 1 | 1 |
| 2 | 25 mg | 25 | 2.5 | 0.57 | 0.057 | | | 4 | 1 | 100 |
| 3 | 1 mg | 100 | 10 | 2.1 | 0.21 | 5 | 5 | 1 | 1 | 1 |
| 4 | 25 mg | 25 | 2.5 | 0.57 | 0.057 | 1.25 | 1.25 | 4 | 1 | 100 |

This is the composition per ml before freeze drying.

Formulations 1 and 3 were made for 1 mg/ml and formulations 2 and 4 for 100 mg/ml APL180. Both concentrations contain 15 mM phosphate buffer pH 7 and 10% trehalose. Formulation 3 and 4 also contains 0.5% TWEEN 80. The solution of 1 mg/ml APL180 is prepared, filled at 1 ml per vial, freeze-dried, and reconstituted with 1 ml water prior to use. The formulation of 100 mg/ml APL180 is prepared at 25 mg/ml APL180 solution, filled at 2 ml per vial, freeze-dried, and reconstituted with 0.5 ml water prior to use. Hence, other ingredients in the solution of 25 mg/ml APL180 are formulated at 25% of the final concentration intended after reconstitution.

Lyophilization cycle is performed as follows:

TABLE 2

| | | | Shelf | |
|---|---|---|---|---|
| | | Time/ | Temperature | Chamber |
| Step | Operation | [hh:mm] | (° C.) | Pressure |
| 1 | Vial loading | As required | 20 | Ambient |
| 2 | Freezing ramp | 01:10 | 20 to −50 | Ambient |
| 3 | Freezing hold | Min. 03:00 | −50 | Ambient |
| | | Max. 70:00 | | |
| 4 | Chamber vacuum | 00:10 | −50 | 0.111 mbar |
| 5 | Primary drying ramp | 06:20 | −50 to −12 | 0.111 mbar |
| 6 | Primary drying hold | 24:00 | −12 | 0.111 mbar |
| 7 | Secondary drying | 06:10 | −12 to 25 | 0.111 mbar |
| | ramp | | | |
| 8 | Secondary drying | 06:00 | 25 | 0.111 mbar |
| | hold | | | |

### Example 2

### Freeze-Dried—Reconstituted Solution Study by Fourier Transform Infrared Spectometry

Freeze-dried compositions of N-Acetyl-L-Asp-L-Trp-L-Phe-L-Lys-L-Ala-L-Phe-L-Tyr-L-Asp-L-Lys-L-Val-L-Ala-L-Glu-L-Lys-L-Phe-L-Lys-L-Glu-L-Ala-L-Phe-Amide (APL180) were made using the ingredients noted in Table 3 below and then reconstituted as noted and tested using Fourier transform infrared spectrometry to determine the % α and % β helices.

TABLE 3

| | | Concentration | | |
|---|---|---|---|---|
| Formula- | | of APL180 | % | % |
| tion # | Ingredient | (mg/ml) | α | β |
| 1 | 1A-10% sucrose in phosphate | 100 | 26 | 40 |
| 2 | buffer | 10 | 27 | 35 |
| 3 | 2A-10% trehalose in phosphate | 100 | 29 | 38 |
| 4 | buffer | 10 | 43 | 28 |
| 5 | 5A-10% sucrose + 0.5% | 100 | 28 | 37 |
| 6 | TWEEN 80 in phosphate buffer | 10 | 28 | 31 |
| 7 | 6A-20% HPbCD in phosphate | 100 | 26 | 38 |
| 8 | buffer | 10 | 24 | 32 |
| 9 | 7A-20% SBEbCD in phosphate | 100 | 24 | 38 |
| 10 | buffer | 10 | 26 | 34 |
| 11 | 1C-10% sucrose in tris buffer | 100 | 22 | 42 |
| 12 | | 10 | 20 | 39 |
| 13 | 1E-10% sucrose in histidine | 100 | 23 | 41 |
| 14 | buffer | 10 | n/a | N/a |
| 15 | In phosphate buffer | 100 | n/a | 42 |
| 16 | | 10 | n/a | 34 |
| 17 | In water | 100 | 19 | 42 |
| 18 | | 10 | 29 | 33 |

### Example 3

### Compositions of 6 mg N-Acetyl-L-Asp-L-Trp-L-Phe-L-Lys-L-Ala-L-Phe-L-Tyr-L-Asp-L-Lys-L-Val-L-Ala-L-Glu-L-Lys-L-Phe-L-Lys-L-Glu-L-Ala-L-Phe-Amide

6 mg APL180 drug product is formulated as sterile, lyophilized powder for intravenous administration. The composition of each vial is provided in Table 4. Each vial is overfilled with 2.2 ml of bulk solution before lyophilization and reconstituted with 2 ml of water for injection (WFI) before administration. Two ml of reconstituted solution will deliver 6 mg APL180.

TABLE 4
Composition of APL180 drug product 6 mg/vial

| Ingredients | Amount per Vial | Rationale for Use |
|---|---|---|
| Trehalose dihydrate | 220.00 mg | Lyo/Cryo-protectant |
| Disodium hydrogen phosphate | 3.461 mg | Buffering agent |
| anhydrous, USP/EP | | |
| Natrium dihydrogen phosphate | 1.342 mg | Buffering agent |
| 2AQ, USP/EP | | |
| Polysorbate 80, NF | 0.220 mg | Surfactant |
| APL180 | 6.600 mg | Active ingredient |

Primary packaging components
vial drawn glass 6 ml/20 mm blow back
rubber stopper 20 mm Daikyo D777-1, V10-F7-3W B2-TR lyo, RS
aluminum flip off PP/AL 20 mm nature/nature

Bulk liquid formulation composition
APL180 is formulated as bulk liquid before being filled into vials and freeze-dried. The composition of the bulk liquid formulation is provided in Table 5.

TABLE 5
Composition of APL 180 bulk liquid
formulation before lyophilization

| Ingredient | Amount per ml | |
|---|---|---|
| Trehalose dihydrate | 100.00 | mg |
| Disodium hydrogen phosphate anhydrous | 1.573 | mg |
| Natrium dihydrogen phosphate 2AQ | 0.610 | mg |
| Polysorbate 80 | 0.100 | mg |
| APL180 | 3.000 | mg |
| Hydrochloric acid 1.0 N | As needed | |
| Sodium hydroxide for injection | As needed | |
| WFI | QS to 1.0 | ml |

Manufacturing Procedures

Preparation of APL180 Formulated Bulk Solution (Compounding)
1. Inspect compounding area and tank/vessel (SS T316L) for cleanliness.
2. Obtain tare weight of compounding vessel
3. Calculate the amount of WFI to add into compounding vessel (about 80% of the total final formulation volume).
4. Add the calculated amount of cool WFI to the compounding vessel.
5. Start a propeller mixer and adjust the speed to a moderate mixing. Add the weighed amounts of:
   a. disodium hydrogen phosphate anhydrous
   b. natrium dihydrogen phosphate 2Aq.
   c. trehalose dihydrate
   d. polysorbate 80
   APL180 will not be added until a, b, c, and d are completely dissolved. Mix for at least 15 minutes. If not dissolved, continue mixing until dissolved by visual inspection.
   e. APL180
6. Add WFI to 98% of the full batch size. Pull a sample for pH. Adjust pH using 1.0 N sodium hydroxide for injection or 1 N HCL if necessary.
7. QS to full batch size with WFI and mix for a minimum of five (5) minutes.
8. Take a sample for IPC testing for appearance, density and pH.
9. Test integrity of two 0.22μ PVDF filters (Millipak 40 or Millipak 20 depending on batch size) using the compounded solution. Discard all the solution used for the integrity testing.
Product specific BPmin=38.5 psi
10. Filter the compounded solution through the integrity tested filters. Discard the first 500 ml of the solution through the second filter.

Lyophilization of APL180 Formulation
11. Aseptically fill 2.2 ml of the bulk solution prepared from Step 10 into clean and sterile 6 ml vial. Discard the first 230 vials (equivalent to 506 ml of solution).
12. Partially insert Daikyo D777-1 lyo rubber stoppers onto the vials.
13. Load the vials into a lyophilizer.
14. Start the lyophilization cycle by following the steps in Table 6.
15. At the end of the cycle, backfill the lyophilization chamber with nitrogen gas with a final chamber pressure of 850±50 mbar, Vials will then be fully stoppered.
16. Cap the inspected vials with Aluminum Flip Off seals.

TABLE 6
Lyophilization Cycle Parameters

| | | | Shelf | |
|---|---|---|---|---|
| | | Time [hh:mm] | Temperature | Chamber |
| Step | Operation | (rate) | (° C.) | Pressure |
| 1 | Vial loading | As required | Ambient | Ambient |
| 2 | Cooling for | As required | 5 | Ambient |
| | uniformity | (1° C./min) | | |
| 3 | Cooling hold | 00:30 | 5 | Ambient |
| 4 | Freezing ramp | 01:30 (0.5° C./min) | −40 | Ambient |
| 5 | Freezing hold | 03:00 | −40 | Ambient |
| 6 | Chamber vacuum | As required | −40 | 0.10 mbar |
| 7 | Primary drying | 00:22 (1° C./min) | −18 | 0.10 mbar |
| | ramp | | | |
| 8 | Primary drying | 32:00 | −18 | 0.10 mbar |
| | hold | | | |
| 9 | Secondary drying | 01:26 (0.5° C./min) | 25 | 0.10 mbar |
| | ramp | | | |
| 10 | Secondary drying | 10:00 | 25 | 0.10 mbar |
| | hold | | | |
| 11 | Ending cycle | 00.20 (1° C./min) | 5 | 0.10 mbar |
| 12 | Preparing for | As required | 5 | 850 mbar |
| | stoppering, | | | |
| | nitrogen | | | |
| | back-filled | | | |
| 13 | Shelf collapse | As required | 5 | 850 mbar |
| 14 | Hold for unload | Min 00:00 | 5 | 850 mbar |
| | | Max 24:00 | | |

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entirety.

What is claimed is:

1. A method of preserving secondary structure during freeze-drying of a peptide comprising the steps of:
(a) admixing trehalose with the peptide in a solution or suspension, said trehalose in an amount sufficient to preserve secondary structure of the peptide; and
(b) freeze-drying the solution or suspension to obtain a peptide composition in which secondary structure has been preserved,
wherein the peptide is selected from the group consisting of N-Acetyl-D-Asp-D-Trp-D-Phe-D-Lys-D-Ala-D-Phe-D-Tyr-D-Asp-D-Lys-D-Val-D-Ala-D-Glu-D-Lys-D-Phe-D-Lys-D-Glu-D-Ala-D-Phe-Amide; N-Acetyl-L-Asp-L-Trp-L-Phe-L-Lys-L-Ala-L-Phe-L-Tyr-L-Asp-L-Lys-L-Val-L-Ala-L-Glu-L-Lys-L-Phe-L-Lys-L-Glu-L-Ala-L-Phe-Amide, D3F, L3F, D5F, L5F, D6F, L6F, D7F and L7F or any pharmaceutically acceptable salt form thereof.

2. The method of claim 1, wherein the peptide is N-Acetyl-L-Asp-L-Trp-L-Phe-L-Lys-L-Ala-L-Phe-L-Tyr-L-Asp-L-Lys-L-Val-L-Ala-L-Glu-L-Lys-L-Phe-L-Lys-L-Glu-L-Ala-L-Phe-Amide or any pharmaceutically acceptable salt form thereof.

3. The method of claim 1 further comprising the step of:
(c) reconstituting the peptide composition to obtain a solution of the peptide in which secondary structure has been preserved.

4. The method of claim 3, wherein the peptide is N-Acetyl-L-Asp-L-Trp-L-Phe-L-Lys-L-Ala-L-Phe-L-Tyr-L-Asp-L-Lys-L-Val-L-Ala-L-Glu-L-Lys-L-Phe-L-Lys-L-Glu-L-Ala-L-Phe-Amide or any pharmaceutically acceptable salt form thereof.

5. The method of claim 1, wherein the secondary structure is an α-helix structure.

6. The method of claim 3, wherein the peptide composition of either step (b) or (c) has a high α-helix content.

7. The method of claim 1, wherein a weight ratio of trehalose to peptide ranging from about 500:0.01 to about 10:200 provides the amount of trehalose sufficient to preserve secondary structure.

8. The method of claim 7, wherein the weight ratio of trehalose to peptide ranges from about 100:0.2 to about 100:30.

9. The method of claim 1, wherein the solution of step (a) further comprises at least one additional excipient.

10. The method of claim 9, wherein the at least one additional excipient is selected from the group consisting of surfactant and buffer, and combinations thereof.

11. The method of claim 10, wherein the surfactant is added prior to the peptide.

12. The method of claim 11, wherein the surfactant is TWEEN 80.

13. The method of claim 12, wherein the TWEEN 80 is present in an amount ranging from about 0.0001 to 10% by weight by volume of the solution of step (a).

14. The method of claim 13, wherein the TWEEN 80 is present in an amount ranging from about 0.005 to 0.1% by weight by volume of the solution of step (a).

15. The method of claim 10, wherein the buffer is selected from the group consisting of sodium phosphospate, potassium phosphate, Tris, citrate, tartrate and histidine.

16. The method of claim 15, wherein the buffer is sodium phosphate buffer which is present in an amount ranging from about 5 mM to 100 mM of the solution of step (a).

17. A freeze-dried composition made according to the method of claim 1.

18. A reconstituted composition made according to the method of claim 3.

19. A freeze-dried composition comprising a peptide selected from the group consisting of N-Acetyl-D-Asp-D-Trp-D-Phe-D-Lys-D-Ala-D-Phe-D-Tyr-D-Asp-D-Lys-D-Val-D-Ala-D-Glu-D-Lys-D-Phe-D-Lys-D-Glu-D-Ala-D-Phe-Amide; N-Acetyl-L-Asp-L-Trp-L-Phe-L-Lys-L-Ala-L-Phe-L-Tyr-L-Asp-L-Lys-L-Val-L-Ala-L-Glu-L-Lys-L-Phe-L-Lys-L-Glu-L-Ala-L-Phe-Amide, D3F, L3F, D5F, L5F, D6F, L6F, D7F and L7F or any pharmaceutically acceptable salt form thereof and an amount of trehalose sufficient to preserve secondary structure of the peptide.

20. The freeze-dried composition of claim 19, wherein the peptide is N-Acetyl-D-Asp-D-Trp-D-Phe-D-Lys-D-Ala-D-Phe-D-Tyr-D-Asp-D-Lys-D-Val-D-Ala-D-Glu-D-Lys-D-Phe-D-Lys-D-Glu-D-Ala-D-Phe-Amide or N-Acetyl-L-Asp-L-Trp-L-Phe-L-Lys-L-Ala-L-Phe-L-Tyr-L-Asp-L-Lys-L-Val-L-Ala-L-Glu-L-Lys-L-Phe-L-Lys-L-Glu-L-Ala-L-Phe-Amide.

21. The freeze-dried composition of claim 20, wherein the peptide is N-Acetyl-L-Asp-L-Trp-L-Phe-L-Lys-L-Ala-L-Phe-L-Tyr-L-Asp-L-Lys-L-Val-L-Ala-L-Glu-L-Lys-L-Phe-L-Lys-L-Glu-L-Ala-L-Phe-Amide.
